(19) United States
(12) Patent Application Publication
(10) Pub. No.: US 2024/0342336 A1
(43) Pub. Date: Oct. 17, 2024

(54) OPTICAL FILTER MODULE AND FOOD WASTE DISPOSAL APPARATUS COMPRISING SAME

(71) Applicant: SAMSUNG ELECTRONICS CO., LTD., Suwon-si (KR)

(72) Inventors: Hongkwan CHO, Suwon-si (KR); Youngchul KO, Suwon-si (KR); Keonpyo KOO, Suwon-si (KR); Dohyun KIM, Suwon-si (KR); Kyoungmok KIM, Suwon-si (KR); Donghyo KIM, Suwon-si (KR)

(73) Assignee: SAMSUNG ELECTRONICS CO., LTD., Suwon-si (KR)

(21) Appl. No.: 18/755,195

(22) Filed: Jun. 26, 2024

Related U.S. Application Data
(63) Continuation of application No. PCT/KR2022/017466, filed on Nov. 8, 2022.

(30) Foreign Application Priority Data
Dec. 29, 2021 (KR) 10-2021-0190918

(51) Int. Cl. A61L 9/20; A61L 9/014; B01D 53/00; B01D 53/04; B01D 53/88; B09B 3/35; B09B 3/38; B09B 3/40; B09B 101/70

(52) U.S. Cl. CPC A61L 9/205; A61L 9/014; B01D 53/007; B01D 53/04; B01D 53/885; B09B 3/35; B09B 3/38; B09B 3/40; A61L 2209/12; A61L 2209/22; B01D 2253/102; B01D 2257/90; B01D 2258/0275; B01D 2259/804; B09B 2101/70

(57) ABSTRACT

A food waste disposal apparatus, according to one embodiment of the present disclosure, comprises: a chamber for accommodating food waste; and a deodorizing device for deodorizing gas inside the chamber. The deodorizing device may comprise: a photocatalyst filter arranged to be inclined; a light source unit for irradiating light having a frequency within a specific range to the photocatalyst filter; and an activated carbon filter in which activated carbon is accommodated so as to deodorize gas passing through the photocatalyst filter.

### CROSS-REFERENCE TO RELATED APPLICATIONS

This application is a continuation of International Application PCT/KR2022/017466, filed Nov. 8, 2022, and claims foreign priority to Korean Application 10-2021-0190918, filed Dec. 29, 2021, which are incorporated herein by reference in their entireties.

### TECHNICAL FIELD

Various embodiments of the disclosure relate to an optical filter module and a food waste disposer including the same.

### BACKGROUND ART

A food waste disposer is a device for treating food waste by grinding/stirring, heating, and drying food waste. Gas containing water vapor generated while heating food waste moves through an exhaust pipe, is deodorized, and then discharged to the outside of the food waste disposer. The gas containing water vapor may be deodorized by passing through an activated carbon filter containing activated carbon that absorbs odor. By photodecomposing the odor inside the water vapor by disposing an optical filter module before the gas containing water vapor flows into the activated carbon filter, it may be possible to enhance deodorization efficiency and increase the life cycle of the activated carbon filter.

Condensate may be generated while the gas containing water vapor passes through the optical filter module. The condensate may form on a light source unit (e.g., a UV-LED) in the optical filter module, in which case the photodecomposition efficiency of the optical filter module may be reduced.

### DETAILED DESCRIPTION OF THE INVENTION

### Technical Solution

Various embodiments of the disclosure may form an inclined structure in the optical filter module to discharge condensate downward.

A food waste disposer according to an embodiment of the disclosure may comprise a chamber configured to accommodate food waste, and a deodorizer. The deodorizer may include a photocatalytic filter that is inclined so as to form an acute angle from a direction of gravity and is configured so that gas from inside the chamber passes through the photocatalytic filter, a light source unit configured to irradiate light having a specific range of frequencies to the photocatalytic filter, and an activated carbon filter configured to deodorize the gas passed through the photocatalytic filter.

According to an embodiment of the disclosure, the light source unit may include a first light source unit configured to irradiate light to an upper surface of the photocatalytic filter and a second light source unit configured to irradiate light to a lower surface of the photocatalytic filter.

According to an embodiment of the disclosure, the deodorizer may further include a cover surface between the light source unit and the photocatalytic filter and formed of a material through which at least a portion of the light irradiated by the light source unit is transmittable to the photocatalytic filter.

According to an embodiment of the disclosure, the cover surface may be inclined so that condensate generated from gas passing through the photocatalytic filter is discharged by gravity along the cover surface.

According to an embodiment of the disclosure, the cover surface may be parallel to the photocatalytic filter.

According to an embodiment of the disclosure, the photocatalytic filter may be inclined by 30 degrees to 60 degrees from the direction of gravity.

According to an embodiment of the disclosure, the light source unit may be spaced apart from the photocatalytic filter by 15 mm to 25 mm.

According to an embodiment of the disclosure, the light source unit may include a plurality of UV-LEDs, and may be disposed to irradiate light in a direction perpendicular to a surface of the photocatalytic filter.

A food waste disposer according to an embodiment of the disclosure may comprise a chamber configured to accommodate food waste, and a deodorizer. The deodorizer may include a photocatalytic filter configured so that gas from inside the chamber passes through the photocatalytic filter, a light source unit configured to irradiate light having a specific range of frequencies to the photocatalytic filter, an activated carbon filter configured to deodorize the gas passed through the photocatalytic filter, and a cover surface between the photocatalytic filter and the light source unit and having a material through which at least a portion of the light irradiated by the light source unit passes to the photocatalytic filter.

According to an embodiment of the disclosure, the cover surface may be inclined from a direction of gravity.

An optical filter module according to an embodiment of the disclosure may comprise a case, a photocatalytic filter in the case, a light source unit configured to irradiate light having a specific range of frequencies to the photocatalytic filter, and a cover surface between the photocatalytic filter and the light source unit to cover the light source unit.

According to an embodiment of the disclosure, the cover surface may be inclined so that condensate generated inside the optical filter module is discharged by gravity along the cover surface.

According to an embodiment of the disclosure, the photocatalytic filter may be inclined so as to form an acute angle from a direction of gravity.

According to an embodiment of the disclosure, the light source unit may be spaced apart from the photocatalytic filter by 15 mm to 25 mm.

According to an embodiment of the disclosure, the case may further include a plurality of heat dissipation holes for discharging heat to outside of the optical filter module.

According to various embodiments proposed in the disclosure, it is possible to prevent performance degradation or failure of the light source unit due to condensed water condensed in the optical filter module.

### BRIEF DESCRIPTION OF DRAWINGS

FIG. 1 is a perspective view illustrating a food waste disposer according to an embodiment of the disclosure;

FIG. 2 is a perspective view illustrating a state in which a cover member of the food waste disposer of FIG. 1 is open;

FIG. 3 is a rear perspective view of the food waste disposer of FIG. 1;

FIG. 4 is a cross-sectional view of the food waste disposer of FIG. 1;

FIG. 5 is a perspective view illustrating a state in which a housing and a cover member are removed from a food waste disposer according to an embodiment of the disclosure;

FIG. 6 is an exploded perspective view of the food waste disposer of FIG. 5;

FIG. 7 is a rear view of the food waste disposer of FIG. 5;

FIG. 8 is a perspective view illustrating a deodorizer according to an embodiment of the disclosure;

FIG. 9 is a cross-sectional view illustrating a deodorizer according to an embodiment of the disclosure;

FIG. 10 is an exploded perspective view illustrating an optical filter module according to an embodiment of the disclosure;

FIG. 11 is a cross-sectional view illustrating an optical filter module according to an embodiment of the disclosure;

FIG. 12A is a side view illustrating a light source unit and a photocatalyst filter;

FIG. 12B is a front view illustrating a light source unit and a photocatalyst filter;

FIG. 13 is a graph illustrating photodecomposition efficiency according to a distance between a light source unit and a photocatalyst filter;

FIG. 14 is a view schematically illustrating an optical filter module according to a second embodiment;

FIG. 15 is a view schematically illustrating an optical filter module according to a third embodiment;

FIG. 16 is a view schematically illustrating an optical filter module according to a fourth embodiment; and

FIG. 17 is a view schematically illustrating an optical filter module according to a fifth embodiment.

### MODE FOR CARRYING OUT THE INVENTION

Embodiments of the present invention are now described with reference to the accompanying drawings in such a detailed manner as to be easily practiced by one of ordinary skill in the art. However, the disclosure may be implemented in other various forms and is not limited to the embodiments set forth herein. The same or similar reference denotations may be used to refer to the same or similar elements throughout the specification and the drawings. Further, for clarity and brevity, no description is made of well-known functions and configurations in the drawings and relevant descriptions.

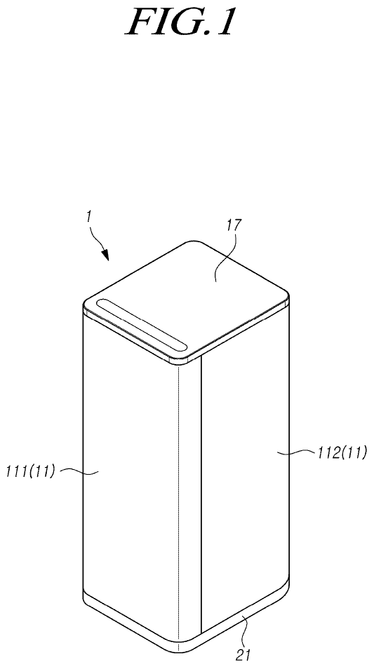

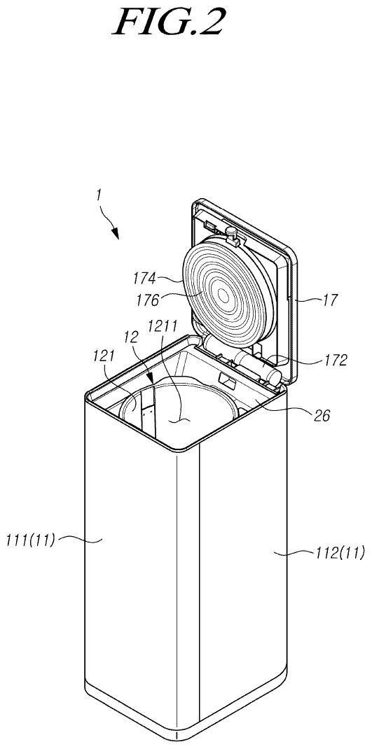

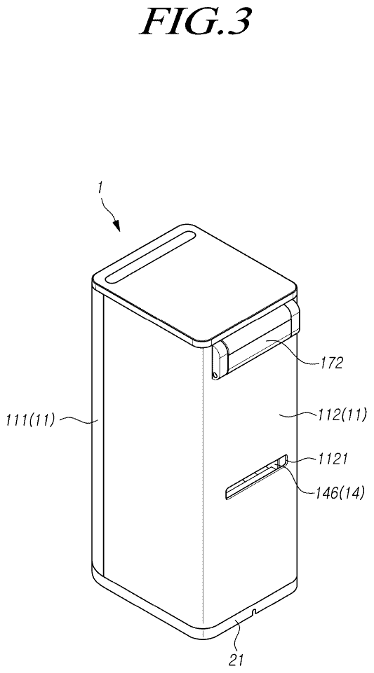

FIG. 1 is a perspective view illustrating a food waste disposer according to an embodiment of the disclosure. FIG. 2 is a perspective view illustrating a state in which a cover member of the food waste disposer of FIG. 1 is open. FIG. 3 is a rear perspective view of the food waste disposer of FIG. 1.

Referring to FIGS. 1 to 3, a food waste disposer 1 according to an embodiment of the disclosure may include a housing 11, a cover member 17, or a treating device 12.

According to an embodiment, the housing 11 may include a front housing 111 or a rear housing 112. The housing 11 may form, e.g., an outer appearance of the food waste disposer 1. The housing 11 may be disposed, e.g., on a lower frame 21 forming the bottom surface of the food waste disposer 1.

The front housing 111 may form, e.g., a front exterior of the food waste disposer 1. The rear housing 112 may form, e.g., a rear exterior of the food waste disposer 1. The front housing 111 may be provided to be detachable from, e.g., the rear housing 112. The user may access various components disposed inside the food waste disposer 1 by separating the front housing 111 or the rear housing 112.

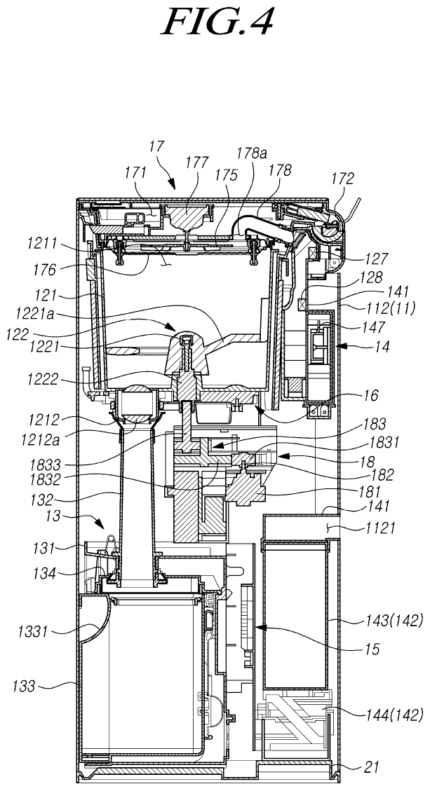

According to an embodiment, the rear housing 112 may include an exhaust hole 1121. The exhaust hole 1121 may be formed to communicate with, e.g., an outlet 146 of a deodorizer (e.g., the deodorizer 14 of FIG. 4). The deodorizer (e.g., the deodorizer 14 of FIG. 4) may be a device for filtering air including the odor generated in the chamber 121 of the treating device 12. The air filtered by the deodorizer (e.g., the deodorizer 14 of FIG. 4) may be discharged to the outside through the exhaust hole 1121.

According to an embodiment, the cover member 17 may include a hinge 172, a treating device cover 174, a convection fan (e.g., the convection fan 175), or a grill 176. The cover member 17 may be disposed to be rotatable with respect to the housing 11 through a hinge 172 connected to the upper frame 26. The treating device cover 174 may be provided to open and close the chamber 121, and may convect air in the chamber 121 by rotating the convection fan 175 in a state in which the chamber 121 is closed. Details of the cover member 17 are described below.

According to an embodiment, the treating device 12 may include a chamber 121 and a stirrer (e.g., the stirrer 122 of FIG. 4). The user may open the cover member 17 to contain food waste to be processed through an opening 1211 of the chamber 121, and then close the cover member 17 again. Food received in the chamber 121 may be crushed or stirred by a stirrer (e.g., the stirrer 122 of FIG. 4). Details of the treating device 12 are described below.

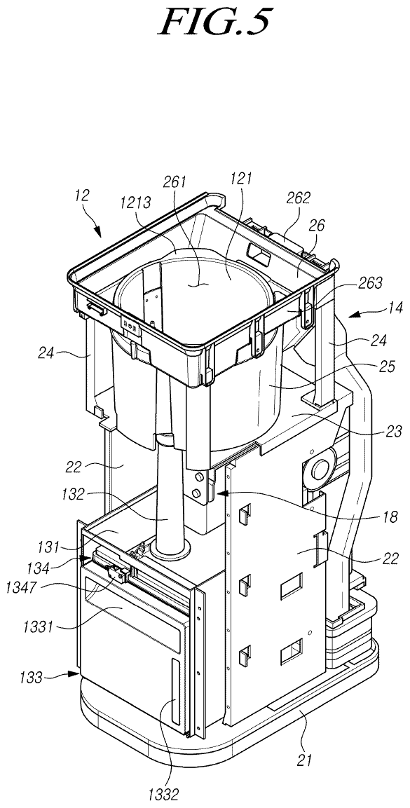

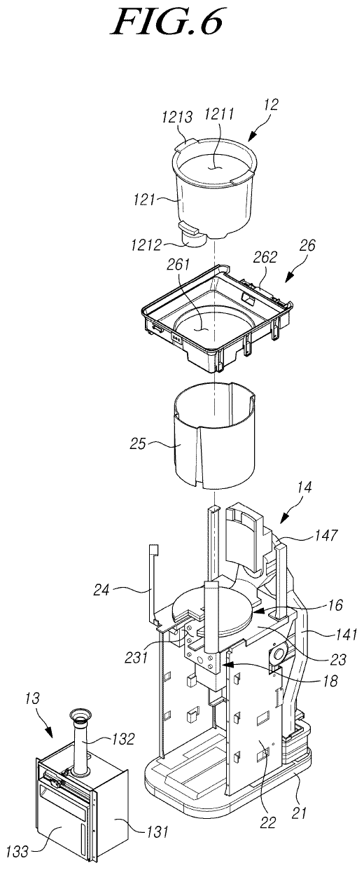

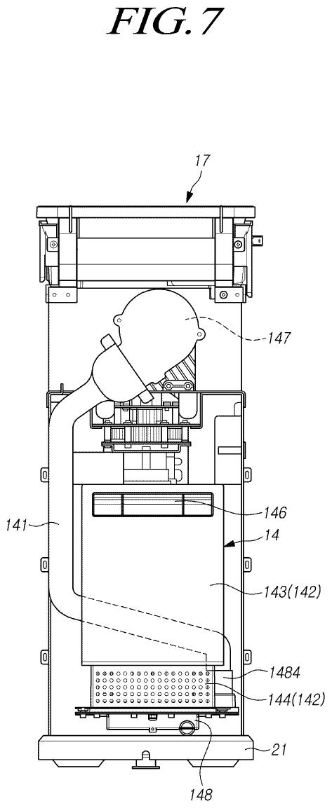

FIG. 4 is a cross-sectional view of the food waste disposer of FIG. 1. FIG. 5 is a perspective view illustrating a state in which a housing and a cover member are removed from a food waste disposer according to an embodiment of the disclosure. FIG. 6 is an exploded perspective view of the food waste disposer of FIG. 5. FIG. 7 is a rear view of the food waste disposer of FIG. 5.

Referring to FIGS. 4 to 6, a food waste disposer 1 according to an embodiment of the disclosure may include a housing 11, a treating device 12, a collecting device 13, a deodorizer 14, a heating device 16, a cover member 17, or a controller 15. The housing 11, the cover member 17, or the treating device 12 illustrated may be identical in whole or part to the housing 11, the cover member 17, or the treating device 12 illustrated in FIGS. 1 to 3.

According to an embodiment, the housing 11 may form an accommodation space in which the components of the food waste disposer 1 are disposed. The housing 11 may be provided to protect, e.g., components disposed in the accommodation space from external shocks.

According to various embodiments, the treating device 12 may include a chamber 121 or a stirrer 122. The treating device 12 may be, e.g., a device for accommodating food waste and grinding, stirring, or heating the food waste.

According to an embodiment, an upper portion of the chamber 121 may be open to form an opening 1211. The opening 1211 may be opened and closed by the cover member 17. The opening 1211 may be, e.g., circular, but is not limited thereto. The chamber 121 may form a space in which food waste may be stirred, heated, or dried. Hereinafter, stirring, heating, or drying food waste is defined as treating the food waste. Food waste may be introduced from an upper portion of the chamber 121.

According to an embodiment, an opening/closing part 1212 may be disposed at a portion of a lower portion of the chamber 121. The opening/closing part 1212 may be provided, e.g., to discharge by-products generated by treating the food waste from the chamber 121. The opening/closing part 1212 may be closed while the food waste is being treated, and may be opened after the food waste treatment is completed. The opening/closing part 1212 may be opened or closed by, e.g., a ball valve 1212a disposed therein, but is not limited thereto. The opening/closing part 1212 may be closed when the food waste disposer 1 does not operate. The by-product may pass through the opening/closing part 1212 and be transferred to a storage container 133 storing the by-product. Here, the by-product may be a portion remaining after food waste treatment is completed in the chamber 121.

According to an embodiment, a handle portion 1213 formed to partially protrude radially outward may be disposed at an upper end of the chamber 121. The user may detach the chamber 121 from the food waste disposer 1 using the handle portion 1213.

According to an embodiment, the stirrer 122 may include a rotation grinder 1221 or a power transmission member 1222. One end of the power transmission member 1222 may be connected to the rotating grinder 1221, and the other end of the power transmission member 1222 may be connected to the driving device 18. The rotating grinder 1221 may be connected to, e.g., the power transmission member 1222 to rotate about the power transmission member 1222. The rotating grinder 1221 may include, e.g., a plurality of blades 1221a. The treating device 12 may grind or stir food waste filling the chamber 121 by rotating the rotating grinder 1221. In other words, the stirrer 122 may grind and stir the food waste in the chamber 121 so that the food waste is evenly heated.

According to an embodiment, the driving device 18 may include a driving motor 181, a shaft 182, or a gear assembly 183. For example, one side of the shaft 182 may be connected to the driving motor 181 and the other side of the shaft 182 may be connected to the gear assembly 183. Specifically, the other side of the shaft 182 may be connected to a first gear 1831 of the gear assembly 183. The shaft 182 may transfer, e.g., the rotational power of the driving motor 181 to the gear assembly 183. The rotational power transferred to the gear assembly 183 may be transferred to the power transmission member 1222.

According to an embodiment, the gear assembly 183 may include a first gear 1831, a second gear 1832, or a third gear 1833, but is not limited thereto. The first gear 1831 may be coupled to, e.g., the shaft 182 and may be disposed to rotate with driving of the driving motor 181. The second gear 1832 may be rotatably disposed to engage with, e.g., the first gear 1831. The third gear 1833 may be rotatably disposed to engage with, e.g., the second gear 1832. The third gear 1833 may be coupled to, e.g., the power transmission member 1222 to transfer the rotational power transferred from the driving motor 181 to the power transmission member 1222.

According to an embodiment, the driving device 18 may transfer rotational power to the power transmission member 1222. The power transmission member 1222 may be disposed to penetrate, e.g., a portion of a lower surface of the chamber 121. For example, the power transmission member 1222 may be disposed so that a portion thereof is positioned inside the chamber 121 and another portion is positioned outside the chamber 121. In other words, among the components connected to the upper and lower ends of the power transmission member 1222, the rotating grinder 1221 may be positioned inside the chamber 121, and the driving device 18 may be positioned outside the chamber 121. The stirrer 122 may be disposed, e.g., under the chamber 121.

According to an embodiment, the heating device 16 may be disposed adjacent to an outer surface of the chamber 121. The heating device 16 may be positioned, e.g., under the chamber 121, but is not limited thereto. The heating device 16 may generate heat, e.g., by heating wires provided therein. When the temperature inside the chamber 121 is increased by the heating device 16, moisture in the food waste may be evaporated and dried. The evaporated water vapor is discharged to the deodorizer 14 to be described below and, after the odor or the like is filtered, is discharged to the outside.

The heating device 16 may be disposed, e.g., between the chamber 121 and an accommodation frame 25 provided to fix the position of the chamber 121. Here, the accommodation frame 25 may be a frame that covers the outside of the treating device 12 or the heating device 16 so that the treating device 12 or the heating device 16 is more stably supported and fixed inside the food waste disposer 1.

According to an embodiment, the cover member 17 may include a hinge 172, a treating device cover 174, a convection fan 175, or a grill 176.

According to an embodiment, the treating device cover 174 may be disposed under the cover member 17 to open and close the opening 1211 above the chamber 121. The treating device cover 174 may have, e.g., a shape corresponding to the shape of the upper surface of the chamber 121. When the treating device cover 174 closes the upper surface of the chamber 121, the air inside the chamber 121 may pass through the discharge pipe 178 of the cover member 17 and flow to the deodorizer 14.

According to an embodiment, the hinge 172 may be disposed at a portion adjacent to an upper end of the chamber 121. The hinge 172 may be positioned, e.g., at a position corresponding to a hinge mounting part 262 of the upper frame 26 to be described below. The hinge 172 may be mounted on, e.g., the hinge mounting part 262 of the upper frame 26. Accordingly, the cover member 17 may be opened and closed by rotating about the hinge 172. The user may open the cover member 17 to put food waste into the chamber 121.

According to an embodiment, the cover member 17 may be provided with an inner space 171 so that components may be disposed therein. For example, the convection fan 175, the fan driver 177, or the discharge pipe 178 may be disposed in the inner space 171.

A portion of the lower portion of the cover member 17 may be formed to be open. Specifically, the treating device cover 174 of the cover member 17 may be formed to have an open lower portion. Accordingly, the lower portion of the treating device cover 174 may be open to allow the flow of air generated by the rotation of the convection fan 175 to be transferred into the chamber 121, forming convection. For example, a grill 176 may be disposed in the open portion of the lower portion of the cover member 17.

According to an embodiment, the convection fan 175 may be disposed in the inner space 171 of the cover member 17. The convection fan 175 may be disposed to rotate about, e.g., a portion of the cover member 17 adjacent to the center of the cover member 17. The food waste disposer 1 may convect air in the chamber 121 by rotating, e.g., the convection fan 175. In other words, when the chamber 121 is heated, the food waste disposer 1 may drive the convection fan 175 to circulate the air in the chamber 121, thereby forming a uniform internal temperature. The convection fan 175 may be rotated, e.g., by a fan driver 177 disposed in the inner space 171 of the cover member 17.

According to an embodiment, the grill 176 may be disposed under the convection fan 175. The grill 176 may be disposed, e.g., on a lower surface of the treating device cover 174. In other words, the grill 176 may be disposed between the chamber 121 and the convection fan 175. A plurality of holes may be formed in the grill 176, and the flow of air generated by the rotation of the convection fan 175 may pass through the plurality of holes and be transferred to the chamber 121. The grill 176 may be fixed to the cover member 17, e.g., by screw coupling.

According to an embodiment, the discharge pipe 178 may be provided so that the discharge port 178a is positioned above the convective fan 175. The discharge port 178a may be positioned above the convection fan 175, outside the convection fan 175. The discharge pipe 178 may be disposed so that the opposite end of the discharge port 178a is connected to the exhaust pipe 141 of the deodorizer 14. In other words, the discharge pipe 178 is a component for connecting the chamber 121 and the exhaust pipe 141 of the deodorizer 14, and may be provided to allow the gas in the chamber 121 to flow to the deodorizer 14.

Although not shown, a locking device (not shown) may be disposed in the cover member 17 or the chamber 121. The locking device may be provided to lock the cover member 17 so that the user may not open the inside while treating the food waste.

According to various embodiments, the collecting device 13 may include a storage container 133, a storage container cover 134, a case 131, or a transfer pipe 132. The collecting device 13 is, e.g., a device for accommodating and storing by-products treated by grinding, stirring, or heating of food waste in the treating device 12 from the chamber 121. The collecting device 13 may have a structure sealed so that stored by-products or odors that may be generated from the by-products do not leak to the outside.

The storage container 133 may include, e.g., a grip portion 1331 or a transparent window 1332. The user may mount or detach the storage container 133 from the case 131 using the grip portion 1331. The transparent window 1332 may be disposed to be visible from the outside when the storage container 133 is mounted on the case 131. The transparent window 1332 may be formed along the height direction of the storage container 133 so that the user may observe the height of the by-product inside. The grip portion 1331 or the transparent window 1332 may be positioned on a surface corresponding to the open surface (e.g., the open surface 1313 of FIG. 7) of the case 131.

The transfer pipe 132 may be, e.g., a component connecting the chamber 121 and the storage container 133. By-products generated by being treated in the chamber 121 may be transferred to the storage container 133 along the transfer pipe 132. The by-products are transferred from the chamber 121 to the storage container 133 in a free-fall manner. The case 131 may be disposed to be surrounded by, e.g., a lower frame 21 or a side frame 22 to be described below. The case 131 may have an open side surface (e.g., the open surface 1313 of FIG. 7) among the side surfaces thereof. The open surface (e.g., the open surface 1313 of FIG. 7) may be, e.g., a surface open for mounting or detaching the storage container 133. The case 131 may be provided with an inner space in which the storage container 133 is mounted. The user may detach the storage container 133 from the case 131 or mount the storage container 133 to the case 131.

The storage container cover 134 may be provided to seal the storage container 133 when the storage container 133 is mounted on the case 131. The storage container cover 134 may be disposed to be fixed to an upper portion in the inner space of the case 131. According to an embodiment, the storage container cover 134 may have a storage amount detector (e.g., the storage amount detector 1341 of FIG. 7) for detecting the amount of the by-product in the storage container 133. The storage amount detector (e.g., the storage amount detector 1341 of FIG. 7) may be, e.g., an optical sensor.

According to various embodiments, the deodorizer 14 may include an exhaust pipe 141, a deodorizing part 142, an outlet 146, or a deodorizing fan 147. The deodorizer 14 is a device that filters and then discharges the odor when the gas containing the odor generated from the food waste received in the chamber 121 is discharged to the outside of the food waste disposer 1.

According to an embodiment, the deodorizing part 142 may include an activated carbon filter 143 or an optical filter module 144. The gas passing through the deodorizing part 142 may be primarily photolyzed and filtered by the optical filter module 144, and then may be secondarily filtered while passing through the activated carbon filter 143. Before the gas introduced from the chamber 121 is filtered by the activated carbon filter 143, the gas may be primarily filtered by the optical filter module 144 to extend the service life of the activated carbon filter 143. The gas filtered by the deodorizing part 142 may be discharged to the discharge port 146. The outlet 146 may be connected to the exhaust hole 1121 of the rear housing 112, and the filtered gas may be discharged to the exhaust hole 1121.

According to an embodiment, a photocatalytic filter may be provided in the optical filter module 144. The photocatalytic filter may be formed of, e.g., titanium dioxide. For example, when light energy in an ultraviolet band is applied to the photocatalytic filter, the strong reducing power of hydrogen peroxide and the strong oxidizing power of the hydroxyl group are generated on the surface thereof, thereby decomposing various contaminants and harmful components in the gas.

For example, activated carbon having a size of 1 mm to 10 mm may be used in the activated carbon filter 143. The activated carbon may deodorize, e.g., the odor of the gas passing through the optical filter module 144.

According to an embodiment, the deodorizing fan 147 may be disposed in the exhaust pipe 141. The deodorizing fan 147 may be provided, e.g., on a flow path in the deodorizer 14 to flow gas passing through the inside of the deodorizer 14.

According to an embodiment, the food waste disposer 1 may further include a temperature detector 127 or a humidity detector 128. The temperature detector 127 or the humidity detector 128 may be provided to directly or indirectly detect, e.g., temperature or humidity in the chamber 121.

According to an embodiment, the temperature detector 127 may be disposed in the deodorizer 14. The temperature detector 127 may be provided, e.g., in the exhaust pipe 141 of the deodorizer 14. Specifically, as illustrated, the temperature detector 127 may be disposed adjacent to the discharge pipe 178 in the exhaust pipe 141 to detect the temperature of the gas immediately after the gas in the chamber 121 passes through the discharge pipe 178 and flows into the exhaust pipe 141. The temperature detector 127 may indirectly detect the temperature in the chamber 121 by detecting the temperature of the gas passing through the discharge pipe 178.

According to an embodiment, the temperature detector 127 may be disposed in the treating device 12. In particular, the temperature detector 127 may be disposed on an upper portion of the chamber 121 to directly detect the temperature in the chamber 121.

According to an embodiment, the humidity detector 128 may be disposed in the deodorizer 14. The humidity detector 128 may be disposed, e.g., in the exhaust pipe 141 of the deodorizer 14. Specifically, the humidity detector 128 may be disposed above the deodorizing fan 147 as illustrated. The humidity detector 128 may indirectly detect the humidity in the chamber 121 by detecting the humidity of the gas in the exhaust pipe 141. According to an embodiment, the humidity detector 128 may be disposed adjacent to the temperature detector 127.

According to an embodiment, the humidity detector 128 may be disposed in the treating device 12. In particular, the humidity detector 127 may be disposed above the chamber 121 to directly detect humidity in the chamber 121.

The temperature detector 127 or the humidity detector 128 may transmit, e.g., information about temperature or humidity to the controller 15. The controller 15 may control operations of devices used to process food waste, such as the stirrer 122, the convection fan 175, the fan driver 177, the driving device 18, or the heating device 16, using the received information about temperature or humidity.

According to an embodiment, the controller 15 may control the overall operation of the electronic components of the food waste disposer 1. In other words, the controller 15 may perform overall control necessary for treating the food waste put into the chamber 121.

For example, the controller 15 may perform control to rotate the stirrer 122 by operating the driving device 18 to crush or stir food waste. For example, the controller 15 may perform control to heat the heating wire in the heating device 16 to heat the inside of the chamber 121. For example, the controller 15 may control the operation of the convection fan 175 to convect the gas in the chamber 121. For example, the controller 15 may perform control to open the opening/closing part 1212 to transfer the remaining by-products to the storage container 133 after the food waste is treated.

The controller 15 may adjust the heat generation intensity of the heating device 16 or the rotation intensity of the convection fan 175 using, e.g., the information about the temperature or humidity in the chamber 121 received from the temperature detector 127 or the humidity detector 128, thereby enhancing the operation efficiency of the food waste disposer 1. Further, the controller 15 may determine whether the storage container 133 is filled with a specific amount or more by-products using a storage amount detector (e.g., the storage amount detector 1341 of FIG. 7) and perform control to notify the user.

The food waste disposer 1 may include a plurality of frames 20 for supporting or fixing components in the housing 11. According to various embodiments, the plurality of frames 20 may include a lower frame 21, a side frame 22, an intermediate frame 23, a supporting frame 24, an accommodation frame 25, or an upper frame 26.

According to an embodiment, the lower frame 21 may form a bottom surface of the food waste disposer 1. The lower frame 21 may support, e.g., the front housing 111 or the rear housing 112. The lower frame 21 may support, e.g., the collecting device 13. The lower frame 21 may support, e.g., a lower end of the side frame 22. Specifically, the lower end of the side frame 22 may be positioned at two opposite ends of the upper surface of the lower frame 21.

According to an embodiment, the side frame 22 may constitute at least a portion of the side surface of the food waste disposer 1. For example, a pair of side frames 22 may be formed on two opposite side surfaces of the food waste disposer 1. The side frame 22 may be disposed to be perpendicular to, e.g., the lower frame 21. The side frames 22 may be provided to surround, e.g., two opposite sides of the collecting device 13.

According to an embodiment, the intermediate frame 23 may be provided, e.g., to partition the placements of the treating device 12 and the collecting device 13 in the food waste disposer 1 or to assist support of the intermediate portion of the food waste disposer 1 to solidify the food waste disposer 1. The intermediate frame 23 may be coupled to, e.g., the upper end of the side frame 22. In other words, the intermediate frame 23 may be supported by the side frame 22. The intermediate frame 23 may be disposed to be parallel to, e.g., the lower frame 21. The heating device 16 or the treating device 12 may be disposed above the intermediate frame 23.

According to an embodiment, the intermediate frame 23 may include a cut-out portion 231 formed as a portion thereof is cut. The cut-out portion 231 may be formed, e.g., in the front. The cut-out portion 231 may be, e.g., a space through which the transfer pipe 132 for transferring by-products from the treating device 12 to the collecting device 13 passes.

According to an embodiment, a space in which the collecting device 13 is received may be formed by the lower frame 21, the side frame 22, and the intermediate frame 23.

According to an embodiment, the supporting frame 24 may be provided to support the surroundings of the treating device 12 of the food waste disposer 1. For example, a plurality of supporting frames 24 may be disposed at corner portions, respectively, of the intermediate frame 23. The number of supporting frames 24 may be, e.g., four, but is not limited thereto. The treating device 12 may be disposed inside the plurality of supporting frames 24.

According to an embodiment, the accommodation frame 25 may be provided to receive the treating device 12. The accommodation frame 25 may be disposed, e.g., between the upper frame 26 and the intermediate frame 23. The accommodation frame 25 may be disposed, e.g., inside a plurality of supporting frames 24. The accommodation frame 25 may be formed, e.g., in a substantially cylindrical shape with open upper and lower portions.

According to an embodiment, the upper frame 26 may be provided to form an upper surface of the food waste disposer 1 when the cover member 17 is opened. According to an embodiment, the upper frame 26 may include an opening 261 or a hinge mounting part 262.

According to an embodiment, the opening 261 may be formed in front of the upper frame 26. The opening 261 may be provided, e.g., to allow the chamber 121 to pass through. Accordingly, a portion of the chamber 121 may be disposed to protrude upward from the upper frame 26. The user may put food waste through the opening 1211 of the chamber 121 protruding upward of the upper frame 26. The shape and size of the opening 261 may correspond to the cross-sectional shape and size of the chamber 121. The opening 261 may be, e.g., circular in shape, but is not limited thereto.

According to an embodiment, the hinge mounting part 262 may be formed behind the upper frame 26. The hinge 172 of the cover member 17 may be mounted on the hinge mounting part 262. Accordingly, the cover member 17 may rotate about the hinge 172 with respect to the upper frame 26.

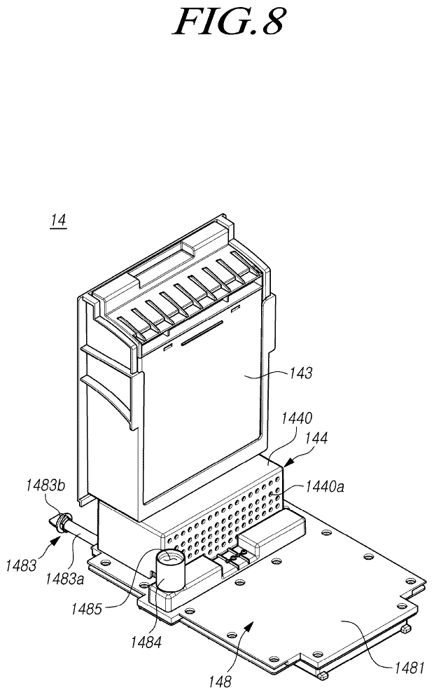

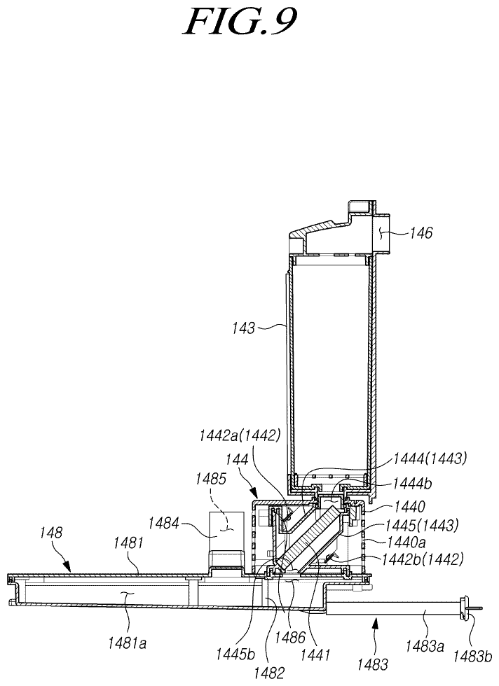

FIG. 8 is a perspective view illustrating a deodorizer according to an embodiment of the disclosure. FIG. 9 is a cross-sectional view illustrating a deodorizer according to an embodiment of the disclosure.

The deodorizer 14 illustrated in FIGS. 8 and 9 may be wholly or partially the same as the deodorizer 14 illustrated in FIGS. 4 to 7. In the deodorizer of FIGS. 8 and 9, the configuration of the exhaust pipe (e.g., the exhaust pipe 141 of FIG. 7) is omitted for convenience of description.

The gas moved from the exhaust pipe 141 may include water vapor. The gas transported from the exhaust pipe 141 may pass through a condenser 148. The condenser 148 may condense at least a portion of the water vapor in the gas before the gas flows into the deodorizing part 142. The gas having reduced moisture may be deodorized by the optical filter module 144 and the activated carbon filter 143 and then discharged to the outlet 146. The deodorization effect of the deodorizing part 142 may be enhanced by condensing a portion of moisture of the gas flowing from the chamber 121 in the condenser 148.

According to an embodiment, the deodorizer 14 may include an exhaust pipe 141, a deodorizing part 142, an outlet 146, a deodorizing fan 147, or a condenser 148. A description of the above-described configuration of the deodorization device 14 is omitted below.

According to an embodiment, the condenser 148 may include a condensing case 1481, a partition wall 1482, a condensate discharge part 1483, an exhaust pipe insertion part 1484, a first inlet 1485, or a second inlet 1445b. The condenser 148 may be disposed between the exhaust pipe 141 and the deodorizing part 142. In other words, the gas flowing from the chamber 121 may pass through the exhaust pipe 141 and then pass through the inside of the condenser 148, and enter the deodorizing part 142.

The condensation case 1481 may include, e.g., a storage space 1481a therein. The storage space 1481a may be, e.g., a space in which condensate is stored. Further, gas introduced from the exhaust pipe 141 to the condenser 148 through the storage space 1481a may flow out to the deodorizing part 142. In other words, the storage space 1481a may also serve as a flow path through which gas flows. According to an embodiment, the condensation case 1481 may be configured as a portion of a lower frame (e.g., the lower frame 21 of FIG. 6). According to an embodiment, the condensation case 1481 may be disposed above the lower frame (e.g., the lower frame 21 of FIG. 6).

According to an embodiment, the partition wall 1482 may be disposed inside the condensation case 1481. In other words, the partition wall 1482 may be disposed in the storage space 1481a. The partition wall 1482 may be a component through which gas may pass. As the gas containing water vapor passes through the partition wall 1482, some water vapor may be condensed and separated from the gas. The partition wall 1482 may form, e.g., a plurality of layers.

The gas containing water vapor may pass through a plurality of partition walls 1482 to the deodorizing part 142. As moisture in the gas is removed by the partition wall 1482, deodorization in the deodorizing part 142 may be more effectively performed.

According to an embodiment, the condensate discharge part 1483 may be formed to extend from a portion of the condensation case 1481. According to an embodiment, the condensate discharge part 1483 may include a condensate discharge pipe 1483a or a discharge lid 1483b. The condensate discharge pipe 1483a may have one end in contact with the condensation case 1481, and the other end having the discharge lid 1483b. The condensate discharge pipe 1483a may be used as, e.g., a passage for discharging condensate in the storage space 1481a. The user may open the discharge lid 1483b to discharge condensate in the storage space 1481a to the outside.

Although not shown, the condenser 148 may further include a water level detector (not shown). For example, the water level detector may be provided to detect the amount of condensate in the storage space 1481a. When it is detected by the water level detector that a predetermined amount of condensate or more fills the storage space 1481a, such a detection signal may be transferred to the controller 15. The controller 15 receiving the detection signal may visually or audibly inform the user that condensate should be discharged from the storage space 1481a.

According to an embodiment, the exhaust pipe insertion portion 1484 may be disposed at an upper end of the condensation case 1481. The exhaust pipe insertion portion 1484 may have, e.g., a shape corresponding to the cross-sectional shape of the exhaust pipe 141 to allow insertion of the exhaust pipe 141.

The first inlet 1485 may refer to a space in which the exhaust pipe insertion part 1484 and the exhaust pipe 141 are connected. The first outlet 1486 may refer to a space in which the condensation case 1481 and the deodorizing part 142 are connected. Specifically, the first outlet 1486 may refer to a space in which the condensation case 1481 and the optical filter module 144 are connected. The gas may flow from the exhaust pipe 141 to the deodorizing part 142 by passing through the first inlet 1485, the storage space 1481a, and the first outlet 1486. The gas flowing into the first outlet 1486 of the condenser 148 may flow into the second inlet 1445b of the optical filter module 144 of the deodorizing part 142.

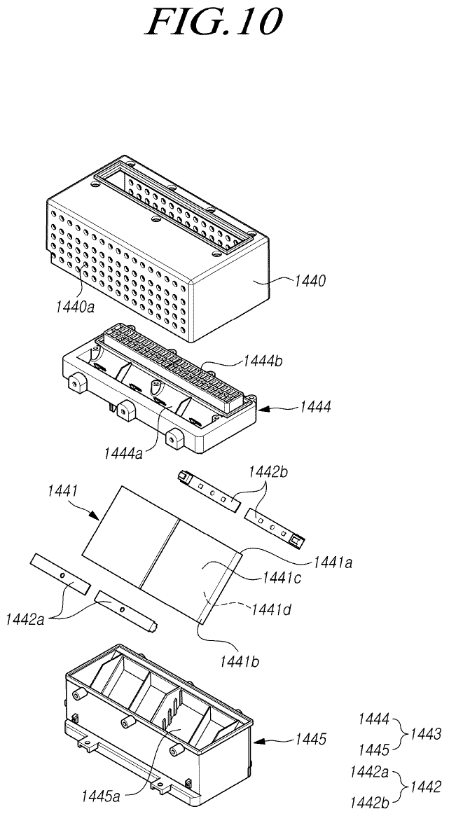

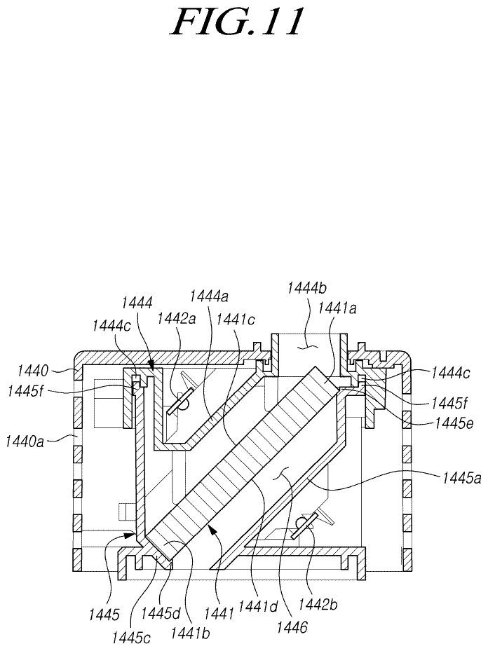

FIG. 10 is an exploded perspective view illustrating an optical filter module according to an embodiment of the disclosure. FIG. 11 is a cross-sectional view illustrating an optical filter module according to an embodiment of the disclosure.

The optical filter module 144 illustrated in FIGS. 10 and 11 may be similar in whole or part to the optical filter module 144 illustrated in FIGS. 4 to 9.

According to an embodiment, the optical filter module 144 may include an outer case 1440, an inner case 1443, a photocatalytic filter 1441, or a light source unit 1442. For example, the outer case 1440 may be provided to protect to protect the components disposed therein from external impact.

According to an embodiment, the outer case 1440 may include a plurality of heat dissipation holes 1440a. For example, the plurality of heat dissipation holes 1440a may discharge the heat generated by heat generation of the light source unit 1442 to the outside. In other words, the heat generated from the light source unit 1442 may be discharged to the outside through the plurality of heat dissipation holes 1440a of the outer case 1440.

According to an embodiment, the photocatalytic filter 1441 may be disposed in the case (the outer case 1440 or the inner case 1443). Specifically, the photocatalytic filter 1441 may be disposed inside a space formed by the upper case 1444 and the lower case 1445. The photocatalytic filter 1441 may be disposed to be inclined. Specifically, the photocatalytic filter 1441 may be disposed to form an acute angle from the direction of gravity. The photocatalytic filter 1441 may be formed of a porous material through which air may pass.

According to an embodiment, the photocatalytic filter 1441 may be supported by the lower case 1445. Specifically, the lower end 1441b of the photocatalytic filter 1441 may be supported by the filter support 1445c of the lower case 1445 formed at one lower side portion (e.g., the lower left portion of FIG. 11). The filter support 1445c may be formed to be inclined to support the photocatalytic filter 1441 to be inclined. In other words, the angle at which the filter support 1445c is disposed may be determined according to the angle formed by the filter support 1445c. The lower end 1441b of the photocatalytic filter 1441 may be stuck to a lower stop jaw 1445d formed at the lower end of the filter support 1445c. The upper end 1441a of the photocatalytic filter 1441 may be stuck to an upper stop jaw 1445e formed at an upper portion on the other side (e.g., the upper right portion of FIG. 11). In other words, the photocatalytic filter 1441 may be seated inside the inner case 1443 by the filter support 1445c, the lower stop jaw 1445d, and the upper stop jaw 1445e of the lower case 1445. The angle formed by the virtual line connecting the lower stop jaw 1445d and the upper stop jaw 1445e and the horizontal surface may be the same as the angle formed by the horizontal surface and the photocatalytic filter 1441. According to an embodiment, a portion of the photocatalytic filter 1441 may be supported or fixed by the upper case 1444. According to an embodiment, the photocatalytic filter 1441 may be supported or fixed only by the lower case 1445. By disposing the photocatalytic filter 1441 to be inclined, a photocatalytic filter 1441 having a wider surface cross-sectional area than when the photocatalytic filter 1441 is disposed in a plane may be disposed. Further, when the photocatalytic filter 1441 of the same size is used, the width of the outer case 1440 may be reduced if it is disposed to be inclined rather than disposed in a plane, so that the optical filter module may be made small and lightweight.

According to an embodiment, the light source unit 1442 may be provided to irradiate light having a specific range of frequencies to the photocatalytic filter 1441. The frequency of the specific range may be UV light. The light source unit 1442 may include, e.g., a plurality of UV-LEDs. The light source unit 1442 may be disposed to be spaced apart from the photocatalytic filter 1441 by a predetermined interval. The light source unit 1442 may be disposed to irradiate light in a direction perpendicular to one surface (e.g., the upper surface 1441c or the lower surface 1441d) of the photocatalytic filter 1441.

According to an embodiment, the light source unit 1442 may include a first light source unit 1442a or a second light source unit 1442b. For example, the first light source unit 1442a may irradiate light to the upper surface 1441c of the photocatalytic filter 1441. For example, the second light source unit 1442b may be disposed to irradiate light to the lower surface 1441d of the photocatalytic filter 1441. For example, the first light source unit 1442a may include four UV-LEDs, but is not limited thereto. The second light source unit 1442b may include four UV-LEDs, but is not limited thereto. The four UV-LEDs disposed in the first light source unit 1442a and the second light source unit 1442b may be disposed to be spaced apart at regular intervals. By irradiating light to two opposite side surfaces (e.g., the upper surface 1441c and the lower surface 1441d) of the photocatalytic filter 1441 using the first light source unit 1442a and the second light source unit 1442b, the photodecomposition efficiency may be enhanced by increasing the activation area of the photocatalytic filter 1441.

According to an embodiment, a cover surface (e.g., a first cover surface 1444a or a second cover surface 1445a) may be disposed between the light source unit 1442 and the photocatalytic filter 1441. The cover surface (e.g., the first cover surface 1444a or the second cover surface 1445a) may be formed of a material through which light is transmitted. The cover surface (e.g., the first cover surface 1444a or the second cover surface 1445a) may be formed of, e.g., polymethyl methacrylate (PMMA) or polycarbonate (PC) material, but is not limited thereto, and may be formed of another material that transmits light and does not transmit liquid that can be used by a person skilled in the art.

According to an embodiment, the first cover surface 1444a may be disposed between the first light source unit 1442a and the upper surface 1441c of the photocatalytic filter 1441. The first cover surface 1444a may be disposed to be inclined. The first cover surface 1444a may be disposed to be parallel to the upper surface 1441c of the photocatalytic filter 1441. The first cover surface 1444a may be formed as a portion of the upper case 1444, but is not limited thereto. According to an embodiment, the first cover surface 1444a may be separately manufactured and coupled to the upper case 1444.

According to an embodiment, the second cover surface 1445a may be disposed between the second light source unit 1442b and the lower surface 1441d of the photocatalytic filter 1441. The second cover surface 1445a may be disposed to be inclined. The second cover surface 1445a may be disposed to be parallel to the lower surface 1441d of the photocatalytic filter 1441. The second cover surface 1445a may be formed as a portion of the lower case 1445, but is not limited thereto. According to an embodiment, the first cover surface 1444a may be separately manufactured and coupled to the lower case 1445.

By having the first cover surface 1444a or the second cover surface 1445a, the light source unit 1442 may irradiate light onto the surface of the photocatalytic filter 1441, and at the same time, prevent condensate generated from the flow path 1446 from forming on the light source unit 1442. When moisture forms on the light source unit 1442, light irradiation performance of the light source unit 1442 may be deteriorated. This may be prevented by disposing the first cover surface 1444a or the second cover surface 1445a. The condensate generated by the gas in the flow path 1446 may form on the first cover surface 1444a or the second cover surface 1445a. In this case, condensate may be flowed down in the direction of gravity by the inclination of the first cover surface 1444a or the second cover surface 1445a, thereby preventing condensate from accumulating on the first cover surface 1444a or the second cover surface 1445a. The condensate flowing down in the direction of gravity may be discharged to the outside through the second inlet 1445b. The condensate discharged through the second inlet 1445b may be stored in a storage space (e.g., the storage space 1481a of FIG. 9) of the condenser (e.g., the condenser 148 of FIG. 9).

According to an embodiment, the inner case 1443 may include an upper case 1444 or a lower case 1445. The inner case 1443 may be disposed inside to be surrounded by the outer case 1440. The upper case 1444 may be disposed to be supported by the lower case 1445. Specifically, as a seating groove 1444c of the upper case 1444 is coupled to the seating protrusion 1445f positioned at the upper end of the lower case 1445, the upper case 1444 may be seated on the lower case 1445. The upper case 1444 and the lower case 1445 may be integrally formed or separated from each other.

According to an embodiment, the lower case 1445 may further include a second inlet 1445b. According to an embodiment, the upper case 1444 may further include a second outlet 1444b. The gas may flow into the second inlet 1445b, flow through the flow path 1446, be filtered by the photocatalytic filter 1441, and then flow out to the second outlet 1444b. The second outlet 1444b may be connected to an activated carbon filter (e.g., the activated carbon filter 143 of FIG. 9).

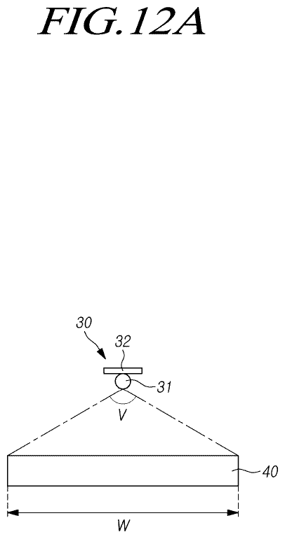

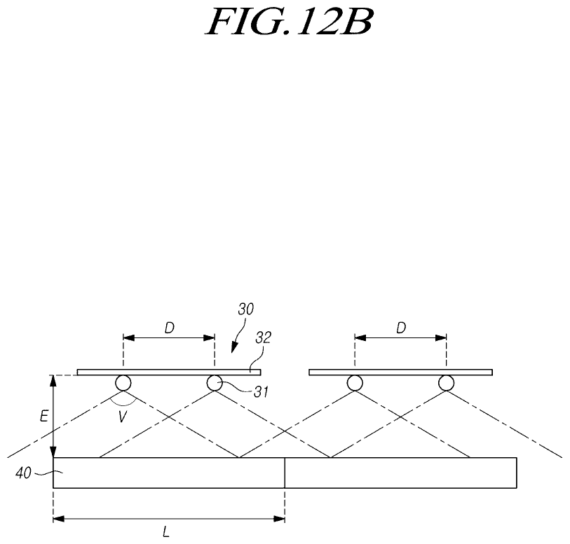

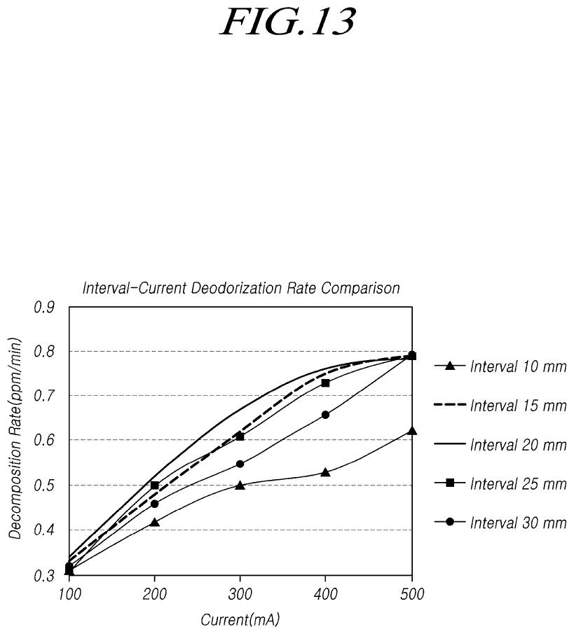

FIG. 12A is a side view illustrating a light source unit and a photocatalyst filter. FIG. 12B is a front view illustrating a light source unit and a photocatalyst filter. FIG. 13 is a graph illustrating photodecomposition efficiency according to a distance between a light source unit and a photocatalyst filter.

The light source unit 30 or the photocatalytic filter 40 illustrated in FIGS. 12A and 12B may be identical in whole or part to the light source unit 1442 or the photocatalytic filter 1441 of the optical filter module 144 illustrated in FIG. 10 or 11. Specifically, the light source unit 30 shown in FIGS. 12A and 12B may be the first light source unit 1442a or the second light source unit 1442b of FIG. 10 or 11.

According to an embodiment, the light source unit 30 may include a substrate 32 or a UV-LED 31. For example, a plurality of UV-LEDs 31 may be disposed to be spaced apart at a constant interval D. The UV-LED 31 may be electrically mounted on the substrate 32. For example, the UV-LED 31 may receive power from the substrate 32 to emit light. The interval D between the plurality of UV-LEDs 31 may be 28 mm, but is not limited thereto.

According to an embodiment, the optical filter module (e.g., the optical filter module 144 of FIG. 4) may include two optical filter units 40. Each optical filter unit 40 may have a substantially square cross section, but is not limited thereto. The size of the optical filter unit 40 may be determined according to a first length W (e.g., a horizontal length) and a second length L (e.g., a vertical length) perpendicular to the first length W. For example, the first length W of the optical filter unit 40 may be 70 mm and the second length L may be 70 mm, but is not limited thereto.

According to an embodiment, two UV-LEDs 31 may be provided to irradiate light of a specific frequency to one surface of one optical filter unit 40. Accordingly, four UV-LEDs 31 may be provided to irradiate light to one surface of the two optical filter units 40, but are not limited thereto. Two UV-LEDs 31 may be mounted on one substrate 32, but are not limited thereto, and all of the four UV-LEDs 31 may be mounted on one substrate 32.

According to an embodiment, the UV-LED 31 may be disposed to be spaced apart from the photocatalytic filter 40 by a predetermined distance E. The decomposition rate may vary depending on the separation distance E between the substrate 32 on which the UV-LED 31 is mounted and the photocatalytic filter 40.

According to an embodiment, the radiation angle V of the light source unit 30 may be determined by at least one of the separation distance D between the UV-LEDs 31 and the separation distance E between the UV-LED 31 and the optical filter unit 40. The separation distance D between the UV-LEDs 31 and the separation distance E between the UV-LED 31 and the optical filter unit 40 may determine a range of the area in which light irradiated by the plurality of UV-LEDs 31 overlaps on one surface of the optical filter unit 40. The radiation angle V of the light source unit 30 may be 120 degrees, but is not limited thereto. The radiation angle V of the light source unit 30 may be determined between 115 degrees and 125 degrees.

FIG. 13 is a graph illustrating the decomposition rate relative to the current value measured for each separation distance E (hereinafter, referred to as the “separation distance E”) between the substrate 32 and the photocatalytic filter 40. Here, the current means a current supplied to the UV-LED 31. The graph of FIG. 13 shows the decomposition rate according to the increase in current when the separation distance E between the substrate 32 and the photocatalytic filter 40 is each of 10 mm, 15 mm, 20 mm, 25 mm, or 30 mm. The illustrated graph may be a graph showing a result when the UV-LED 31 having a radiation angle V of 120 degrees irradiates light to the photocatalytic filter 40 having a first length W of 70 mm and a second length L of 70 mm.

Referring to the illustrated graph, the decomposition rate increases as the current increases regardless of the separation distance E. However, when the same current is supplied to the UV-LED 31, a difference in the decomposition rate may occur according to the separation distance E. For example, when the current supplied to the UV-LED 31 is 200 mA, the decomposition rate may decrease in the order that the separation distance E is 20 mm, 25 mm, 15 mm, 30 mm, and 10 mm. In other words, when the separation distance E is 20 mm, the decomposition rate may be the highest. Referring to the graph shown, when the current supplied to the UV-LED 31 is 100 mA to 400 mA, and when the separation distance E is 20 mm within the corresponding range, the decomposition rate is the highest. As the separation distance E gets away from or closer to 20 mm, the decomposition rate gradually decreased. When the separation distance E is 20 mm, the UV-LED 31 may irradiate light having a range of up to 69.2 horizontally mm and up to 69.2 mm vertically to the surface of the photocatalytic filter 40. In other words, when the separation distance E is 20 mm, most of the light generated by the UV-LED 31 may be irradiated to the surface of the photocatalytic filter 40 having a first length W of 70 mm and a second length L of 70 mm. For efficient gas decomposition, the light source unit 30 and the photocatalytic filter 40 may be disposed so that the separation distance E is 15 mm to 25 mm. However, this is exemplary, and the range of the separation distance E for efficient gas decomposition may vary depending on the size (e.g., the first length W or the second length L) of the photocatalytic filter 40 and the radiation angle V of the UV-LED 31. Specifically, when the size (e.g., the first length W or the second length L) of the photocatalytic filter 40 increases, the range of the separation distance E for efficient gas decomposition may increase. Further, when the radiation angle V of the UV-LED 31 is larger than 120 degrees, the range of the separation distance E for efficient gas decomposition may be reduced.

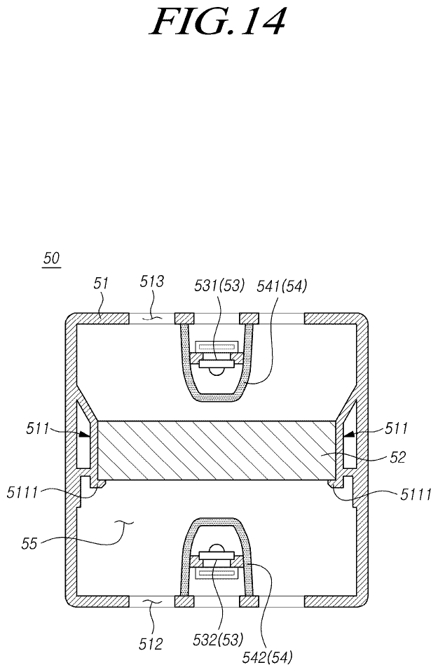

FIG. 14 is a view schematically illustrating an optical filter module according to a second embodiment.

The optical filter module 50 according to the second embodiment shown in FIG. 14 may replace the optical filter module 144 of the deodorizer 14 of FIGS. 8 and 9. In other words, the third inlet 512 of the illustrated optical filter module 50 may correspond to the second inlet 1445b of FIG. 9, and the third outlet 513 of the illustrated optical filter module 50 may be disposed between the condenser 148 and the activated carbon filter 143 to correspond to the second outlet 1444b of FIG. 9.

According to an embodiment, the optical filter module 50 according to the second embodiment may include a case 51, a photocatalytic filter 52, a light source unit 53, a light source unit cover 54, or a flow path 55. Although not shown, the case 51 may be disposed so that the outside is surrounded by the outer case 1440 of FIG. 9. For example, the case 51 may be formed so that the third inlet 512 through which gas flows in and the third outlet 513 through which gas flows out are open. A flow path 55 provided to allow gas to flow may be formed inside the case 51. The illustrated case 51 is integrally configured, but is not limited thereto, and may be divided into an upper case and a lower case.

According to an embodiment, the photocatalytic filter 52 may be fixedly disposed inside the case 51. The photocatalytic filter 52 may be formed of a porous material through which gas may pass. The photocatalytic filter 52 may be fixed and disposed by a fixing portion 511 formed inside the side surface of the case 51. Specifically, the photocatalytic filter 52 may be fixed by spanning the stop jaw 5111 protruding inward from the fixing portion 511. The photocatalytic filter 52 shown is disposed horizontally, but is not limited thereto.

According to an embodiment, the light source unit 53 may be disposed to irradiate the surface of the photocatalytic filter 52. For example, the light source unit 53 may include a UV-LED. For example, the light source unit 53 may be disposed to irradiate one surface of the photocatalytic filter 52 in the vertical dryer. For example, the light source unit 53 may include a first light source unit 531 spaced apart from the upper side of the photocatalytic filter 52 or a second light source unit 532 spaced apart from the lower side of the photocatalytic filter 52. For example, the first light source unit 531 may irradiate light to the upper surface of the photocatalytic filter 52. For example, the second light source unit 532 may irradiate light to the lower surface of the photocatalytic filter 52.

According to an embodiment, the light source unit cover 54 may be provided to shield the light source unit 53 from a gas such as external water vapor. The light source cover 54 may be at least partially formed of a material through which light passes. For example, the light source cover 54 may have an end portion adhered and fixed to the upper or lower surface of the case 51, but is not limited thereto. The light source cover 54 may be shaped substantially as “U”, but is not limited thereto. When shaped as “U”, the light source unit cover 54 may be disposed so that the open portion is adjacent to the inside of the upper surface or the lower surface of the case 51. The light source cover 54 may have a portion through which the light of the light source unit 53 passes, which is formed as a flat surface to prevent refraction of light, and the remaining portion formed as a curved surface. By providing the light source cover 54, it is possible to prevent a failure or performance degradation in the light source 53 due to condensate that may occur on the flow path 55.

According to an embodiment, the light source unit cover 54 may include a first light source unit cover 541 covering the first light source unit 531 or a second light source unit cover 542 covering the second light source unit 532.

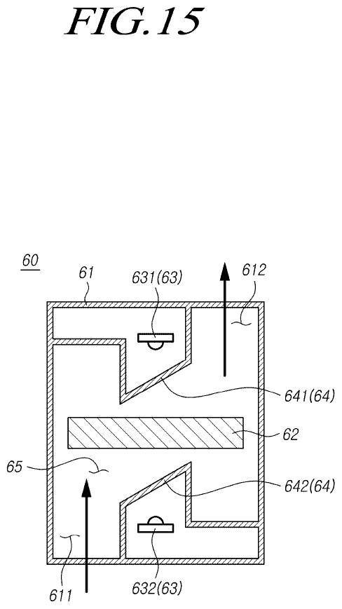

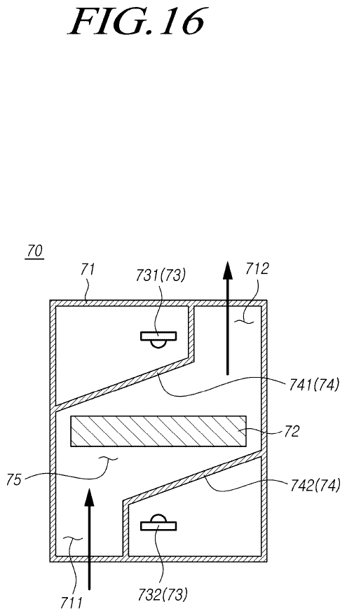

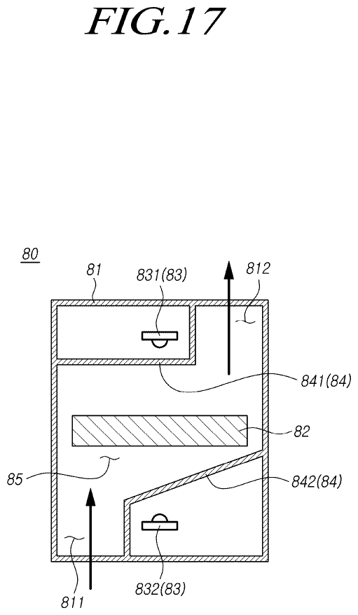

FIG. 15 is a view schematically illustrating an optical filter module according to a third embodiment. FIG. 16 is a view schematically illustrating an optical filter module according to a fourth embodiment. FIG. 17 is a view schematically illustrating an optical filter module according to a fifth embodiment.

Hereinafter, FIGS. 15 to 17 are schematic diagrams for describing an arrangement relationship between the light source unit 63, 73, or 83, the cover surface 64, 74, or 84, and the photocatalytic filter 62, 72, or 82 according to various embodiments. For other specific coupling relationships, FIG. 11 or 14 may be referred to.

The optical filter module 60 according to the third embodiment shown in FIG. 15 may replace the optical filter module 144 of the deodorizer 14 of FIGS. 8 and 9. In other words, it may be disposed between the condenser 148 and the activated carbon filter 143 so that the fourth inlet 611 of the illustrated optical filter module 60 corresponds to the second inlet 1445b of FIG. 9, and the fourth outlet 612 of the illustrated optical filter module 60 corresponds to the second outlet 1444b of FIG. 9.

According to an embodiment, the optical filter module 60 according to the third embodiment may include a case 61, a photocatalytic filter 62, a light source unit 63, a cover surface 64 or a flow path 65. Although not shown, the case 61 may be disposed so that the outside is surrounded by the outer case 1440 of FIG. 9. For example, the case 61 may be formed so that the fourth inlet 611 through which gas flows in and the fourth outlet 612 through which gas flows out are open. A flow path 65 provided to allow gas to flow may be formed inside the case 61. The illustrated case 61 is integrally configured, but is not limited thereto, and may be divided into an upper case and a lower case.

According to an embodiment, the photocatalytic filter 62 may be disposed inside the case 61. The photocatalytic filter 62 may be formed of a porous material through which gas may pass. The photocatalytic filter 52 shown is disposed horizontally, but is not limited thereto.

According to an embodiment, the light source unit 63 may be disposed to irradiate the surface of the photocatalytic filter 62. For example, the light source unit 63 may include a UV-LED. For example, the light source unit 63 may be disposed to irradiate one surface of the photocatalytic filter 62 in the vertical dryer. For example, the light source unit 63 may include a first light source unit 631 spaced apart from the upper side of the photocatalytic filter 62 or a second light source unit 632 spaced apart from the lower side of the photocatalytic filter 62. For example, the first light source unit 631 may irradiate light to the upper surface of the photocatalytic filter 62. For example, the second light source unit 632 may irradiate light to the lower surface of the photocatalytic filter 62.

According to an embodiment, the cover surface 64 may be disposed between the light source unit 63 and the photocatalytic filter 62. The cover surface 64 may be at least partially formed of a material through which light passes. For example, at least a portion of the cover surface 64 may be formed to be inclined. In other words, the inclined surface of the cover surface 64 may be disposed to form an acute angle from the photocatalytic filter 62. The cover surface 64 may be formed to be inclined only with respect to a portion through which light of the light source unit 63 passes. When condensate is generated in the flow path 65 and formed on the cover surface 64, light may be refracted, deteriorating irradiation performance of the light source unit 63. As the portion of the cover surface 64 through which the light of the light source unit 63 passes is formed to be inclined, the condensate may be led to flow in the direction of gravity. The portion of the cover surface 64 through which the light of the light source unit 63 does not pass may be formed in a direction perpendicular or horizontal to the photocatalytic filter 62.

According to an embodiment, the cover surface 64 may include a first cover surface 641 disposed between the first light source unit 631 and the photocatalytic filter 62, or a second cover surface 641 disposed between the second light source unit 632 and the photocatalytic filter 62.

The optical filter module 70 according to the fourth embodiment shown in FIG. 16 may replace the optical filter module 144 of the deodorizer 14 of FIGS. 8 and 9. In other words, it may be disposed between the condenser 148 and the activated carbon filter 143 so that the fifth inlet 711 of the optical filter module 70 illustrated corresponds to the second inlet 1445b of FIG. 9, and the fifth outlet 712 of the optical filter module 70 illustrated corresponds to the second outlet 1444b of FIG. 9.

According to an embodiment, the optical filter module 70 according to the fourth embodiment may include a case 71, a photocatalytic filter 72, a light source unit 73 (e.g., the first light source unit 731 or the second light source unit 732), a cover surface 74, or a flow path 75.

The description of the case 71, the light source unit 73, or the flow path 75 among the illustrated components is described above with reference to FIG. 15, and a detailed description thereof is omitted.

According to an embodiment, the cover surface 74 may include a first cover surface 741 or a second cover surface 742. The cover surface 74 may be formed of a material through which light may pass. For example, the cover surface 74 may be disposed so that condensate or air is not introduced into the light source unit 73.

The first cover surface 741 may be disposed between the first light source unit 731 and the photocatalytic filter 72. The first cover surface 741 may be disposed under the upper surface of the case 71. Specifically, the first cover surface 741 may extend vertically downward from a portion of the upper surface of the case 71 adjacent to the fifth outlet 712, and then obliquely extend downward to the side surface of the case 71 to cover the first light source unit 731. Accordingly, the condensate formed on the surface of the first cover surface 741 may fall to the fifth inlet 711 along the inclined direction.

For example, the second cover surface 742 may be disposed between the second light source unit 732 and the photocatalytic filter 72. The second cover surface 742 may be disposed above the lower surface of the case 71. Specifically, the second cover surface 742 may extend vertically upward from a portion of the lower surface of the case 71 adjacent to the fifth inlet 711 and then obliquely extend upward to the side surface of the case 71 to cover the second light source unit 732. Accordingly, the condensate formed on the surface of the second cover surface 742 may fall to the fifth inlet 711 along the inclined direction.

The optical filter module 80 according to the fifth embodiment shown in FIG. 17 may replace the optical filter module 144 of the deodorizer 14 of FIGS. 8 and 9. In other words, it may be disposed between the condenser 148 and the activated carbon filter 143 so that the sixth inlet 811 of the illustrated optical filter module 80 corresponds to the second inlet 1445b of FIG. 9, and the sixth outlet 812 of the illustrated optical filter module 80 corresponds to the second outlet 1444b of FIG. 9.

According to an embodiment, the optical filter module 80 according to the fifth embodiment may include a case 81, a photocatalytic filter 82, a light source unit 83 (e.g., the first light source unit 831 or the second light source unit 832), a cover surface 84, or a flow path 85. The description of the case 81, the light source unit 83, or the flow path 85 among the illustrated components is described above with reference to FIG. 15, and a detailed description thereof is omitted.

According to an embodiment, the cover surface 84 may include a first cover surface 841 or a second cover surface 842. The second cover surface 842 may correspond to the second cover surface 742 of FIG. 16. Therefore, a description of the second cover surface 842 is omitted.

The first cover surface 841 may be disposed between the first light source unit 731 and the photocatalytic filter 82. The first cover surface 841 may be disposed under the upper surface of the case 81. Specifically, the first cover surface 841 may extend vertically downward from a portion of the upper surface of the case 81 adjacent to the sixth outlet 812, and then horizontally extend to a side surface of the case 81 to cover the first light source unit 831. Since the condensate falls in the direction of gravity, the first cover surface 841 may be formed horizontally, unlike FIG. 16. Further, since the first cover surface 841 has a horizontal surface disposed between the first light source unit 831 and the photocatalytic filter 82, light generated from the first light source unit 831 may be irradiated to the photocatalytic filter 82 without being refracted.

The terms as used herein are provided merely to describe some embodiments thereof, but are not intended to limit the disclosure. As used herein, the singular forms “a,” “an,” and “the” are intended to include the plural forms as well, unless the context clearly indicates otherwise. As used herein, each of such phrases as “A or B,” “at least one of A and B,” “at least one of A or B,” “A, B, or C,” “at least one of A, B, and C,” and “at least one of A, B, or C,” may include all possible combinations of the items enumerated together in a corresponding one of the phrases. As used herein, the term ‘and/or’ should be understood as encompassing any and all possible combinations by one or more of the enumerated items. As used herein, the terms “include,” “have,” and “comprise” are used merely to designate the presence of the feature, component, part, or a combination thereof described herein, but use of the term does not exclude the likelihood of presence or adding one or more other features, components, parts, or combinations thereof. As used herein, the terms “first” and “second” may modify various components regardless of importance and/or order and are used to distinguish a component from another without limiting the components.

As used herein, the terms “configured to” may be interchangeably used with the terms “suitable for,” “having the capacity to,” “designed to,” “adapted to,” “made to,” or “capable of” depending on circumstances. The term “configured to” does not essentially mean “specifically designed in hardware to.” Rather, the term “configured to” may mean that a device can perform an operation together with another device or parts. For example, a ‘device configured (or set) to perform A, B, and C’ may be a dedicated device to perform the corresponding operation or may mean a general-purpose device capable of various operations including the corresponding operation.

Meanwhile, the terms “upper side”, “lower side”, and “front and rear directions” used in the disclosure are defined with respect to the drawings, and the shape and position of each component are not limited by these terms.

In the disclosure, the above-described description has been made mainly of specific embodiments, but the disclosure is not limited to such specific embodiments, but should rather be appreciated as covering all various modifications, equivalents, and/or substitutes of various embodiments.

1. A food waste disposer, comprising:
a chamber configured to accommodate food waste; and
a deodorizer including:
a photocatalytic filter that is inclined so as to form an acute angle from a direction of gravity,
a light source unit configured to irradiate light having a specific range of frequencies to the photocatalytic filter, and
an activated carbon filter configured to deodorize the gas passed through the photocatalytic filter.

2. The food waste disposer of claim 1, wherein the light source unit includes a first light source unit configured to irradiate light to an upper surface of the photocatalytic filter and a second light source unit configured to irradiate light to a lower surface of the photocatalytic filter.

3. The food waste disposer of claim 1, wherein the deodorizer further includes a cover surface between the light source unit and the photocatalytic filter and formed of a material through which light is transmitted.

4. The food waste disposer of claim 3, wherein the cover surface is inclined so that condensate generated from gas passing through the photocatalytic filter is discharged by gravity along the cover surface.

5. The food waste disposer of claim 3, wherein the cover surface is parallel to the photocatalytic filter.

6. The food waste disposer of claim 1, wherein the photocatalytic filter is inclined by 30 degrees to 60 degrees from the direction of gravity.

7. The food waste disposer of claim 1, wherein the light source unit is spaced apart from the photocatalytic filter by 15 mm to 25 mm.

8. The food waste disposer of claim 1, wherein the light source unit includes a plurality of UV-LEDs, and is disposed to irradiate light in a direction perpendicular to a surface of the photocatalytic filter.

9. A food waste disposer, comprising:
a chamber configured to accommodate food waste; and
a deodorizer including:
a photocatalytic filter configured so that gas from inside the chamber passes through the photocatalytic filter,
a light source unit configured to irradiate light having a specific range of frequencies to the photocatalytic filter,
an activated carbon filter configured to deodorize the gas passed through the photocatalytic filter, and
a cover surface between the photocatalytic filter and the light source unit and having a material through which at least a portion of light passes.

10. The food waste disposer of claim 9, wherein the cover surface is inclined from a direction of gravity.

11. An optical filter module, comprising:
a case;
a photocatalytic filter in the case;
a light source unit configured to irradiate light having a specific range of frequencies to the photocatalytic filter; and
a cover surface between the photocatalytic filter and the light source unit to cover the light source unit.

12. The optical filter module of claim 11, wherein the cover surface is inclined so that condensate generated inside the optical filter module is discharged by gravity along the cover surface.

13. The optical filter module of claim 11, wherein the photocatalytic filter is inclined so as to form an acute angle from a direction of gravity.

14. The optical filter module of claim 11, wherein the light source unit is spaced apart from the photocatalytic filter by 15 mm to 25 mm.

15. The optical filter module of claim 11, wherein the case further includes a plurality of heat dissipation holes for discharging heat to outside of the optical filter module.
